(19) United States
(12) Patent Application Publication
(10) Pub. No.: US 2019/0388508 A1
(43) Pub. Date: Dec. 26, 2019

(54) PROGRANULIN AND PROGRANULIN DERIVATIVES IN TREATING IMPAIRED FRACTURE HEALING

(71) Applicant: NEW YORK UNIVERSITY, New York, NY (US)

(72) Inventors: Chuanju LIU, New York, NY (US); Thomas A. EINHORN, New York, NY (US); Jianlu WEI, New York, NY (US)

(73) Assignee: NEW YORK UNIVERSITY, New York, NY (US)

(21) Appl. No.: 16/476,899

(22) PCT Filed: Jan. 19, 2018
(86) PCT No.: PCT/US2018/014324

§ 371 Date: Jul. 10, 2019

Related U.S. Application Data
(60) Provisional application No. 62/448,013, filed on Jan. 19, 2017.

(51) Int. Cl. A61K 38/18

(52) U.S. Cl. CPC A61K 38/18

(57) ABSTRACT

About 5-10% of all fractures result in delayed or impaired healing. Impaired bone fracture is difficult for the patient, and is very expensive for the medical system. The present disclosure provides methods of treating impaired bone fracture healing comprising administration of an effective amount of progranulin or progranulin derivatives to a subject suffering from delayed or impaired bone fracture healing. The disclosure also provides medical compositions and formulations comprising progranulin or progranulin derivatives to be used in the treatment of impaired bone fracture healing.

### CROSS REFERENCE TO RELATED APPLICATION

This application claims the benefit of priority from U.S. Provisional Application No. 62/448,013, filed Jan. 19, 2017, the entire contents of which are incorporated herein by reference.

### STATEMENT REGARDING FEDERALLY SPONSORED RESEARCH OR

DEVELOPMENT

This invention was made with government support under Grant Nos.: R01AR062207; R01AR061484; R56AI100901 awarded by the National Institutes of Health (NIH). The government has certain rights in the invention.

### BACKGROUND ART

In the U.S. alone about 8 million bone fractures occur annually. About 5-10% of these bone fractures experience delayed or impaired healing which brings a huge additional cost to the healthcare industry. In normal bone fractures, a certain amount of time is required before bone healing can be expected to occur. This normal time may vary according to age, the bone involved, the level of the fracture, and associated soft tissue injury. Delayed union, by definition, is present when an adequate period of time has elapsed since the initial injury without achieving bone union, taking into account the above variables. Classically the factors that affect fracture healing can be classified in two broad categories: local factors and systemic factors.

Local factors that may cause impaired healing include problems such as inadequate reduction (reduction is defined as a surgical procedure to restore a fracture or dislocation to the correct alignment), inadequate immobilization, loss of blood supply, too much energy absorption (e.g. energy absorbed by the broken bone during an automobile accident), periosteal and soft tissue vitality or interposition, and infection.

Inadequate reduction of a fracture, regardless of its cause, may be a prime reason for delayed union or nonunion. It usually leads to instability or poor immobilization. In addition, inadequate reduction may be caused by superimposition of soft tissues through the fracture area, which may delay healing. Soft tissue disruption usually leads to loss of vascular supply at the fracture site. In well-muscled areas, this vascular supply may return quickly. In other areas where little muscle is present, this vascular supply may not return.

Inadequate immobilization may result in biomechanical as well as physiologic problems associated with fracture healing.

Systemic factors that cause impaired bone fracture healing include, but are not limited to, age, nutrition, diseases, medications and cigarette smoking. Diseases such as metabolic diseases (e.g. diabetes, osteomalacia, Paget's hypothyroidism), renal function diseases, and Cushing's disease may cause a delay or impairment in fractured bone healing. In addition, medications such as glucocorticoids, bisphosphonates, Nonsteroidal anti-inflammatory drugs (NSAIDS) such as COX-2 inhibitors are also known to cause impaired bone fracture healing in patients.

Cigarette smoking has also been implicated as a risk factor for impaired fracture healing. Smoking has been shown to place patients at risk for increased time to union and complications. Previous smoking history also appears to increase the risk of osteomyelitis (infection and inflammation of the bone and bone marrow) and increased time to union.

The treatment of fractures and large bone defects is a significant challenge. Chondrogenesis and endochondral ossification play a critical role in the healing of these defects, and certain molecules can help to promote bone repair. Bone morphogenetic proteins (BMPs), for example, such as BMP2, induce bone formation and have been applied clinically for fracture healing.

The role of TNF in impaired fracture healing

TNFα-TNFR signaling has received great attention owing to its position at the apex of the pro-inflammatory cytokine cascade and its dominance in the pathogenesis of various disease processes. Several diseases, e.g. diabetes, have been shown to enhance expression of TNFα which is responsible for resorption of mineralized cartilage and bone. Animals with elevated TNFα levels due to diabetes or rheumatoid arthritis demonstrate impaired fracture healing, including smaller calluses and loss of cartilage. Chondrocytes in particular are sensitive to TNFα-induced apoptosis, as BMP-induced stem cells with a chondrogenic phenotype undergo apoptosis in the presence of TNFα, whereas undifferentiated mesenchymal cells do not TNFα plays a key role in delaying fracture healing in diabetes, in part, by driving chondrocyte apoptosis. Additionally, TNFα appears to play a more prominent role in fracture healing in diabetes than in normoglycemic animals. Thus, antagonizing the TNFα inflammatory pathway may be a key strategy in the management of diabetic and other types of impaired fracture healing.

TNFRI and TNFR2: Critical inflammatory signaling in impaired fracture healing

TNFα and its two receptors, TNFR1 and TNFR2, have been extensively studied for their role in bone repair and fracture healing. TNFa is an inflammatory molecule and acts to enhance bone resorption and inhibit osteogenesis, which is largely mediated through its interaction with TNFR1. On the contrary, TNFR2 is believed to have an anti-inflammatory effect, and plays an important role in bone regeneration. In fact, TNFR2 knockout mice have been shown to have a reduced capacity for bone regeneration.

Progranulin (PGRN)

Progranulin (PGRN), also known as granulin-epithelin precursor (GEP), is a growth factor with multiple biological functions including anti-inflammation and immune regulations Man J. et al. (2013), J Leukoc Biol, 93: 199-208.). PGRN contains seven-and-a-half repeats of a cysteine-rich motif (CX5-6CX5CCX8CCX6CCXDX2HCCPX4CX5-6C) in the order P-G-F-B-A-C-D-E, where A-G are full repeats and P is the half-motif, and which forms a unique “beads-on-a-string” structure (Hrabal R. et al., (1996), Nature Structural Biology, 3: 747-752). PGRN was reported to bind to TNF receptors (TNFR) through three individual and separate binding domains involving granulin A, C and F plus adjacent linkers (Tang W. et al. (2011), Science, 332: 478-484.). PGRN is abundantly expressed in epithelial cells, immune cells, and chondrocytes. PGRN is an important regulator of cartilage development and degradation. The human mRNA sequence of PGRN is shown as SEQ ID NO: 1; and the human amino acid sequence of PGRN is shown as SEQ ID NO: 2.

Atsttrin (Antagonist of TNF/TNFR Signaling via Targeting to TNF Receptors) is an engineered progranulin derivative composed of half units of granulins A, C and F plus linkers P3, P4 and P5 that appears to be the “minimal” engineered molecule retaining affinity to TNFR (Tang W. et al. (2011), Science, 332: 478-484; Liu C J et al., (2012), Pharmacology & Therapeutics, 133: 124-132; Liu C J. (2011), FEBS Letters, 585: 3675-3680.) Atsttrin was reported to selectively bind to TNFR and inhibited the binding of TNFα to TNFR in vitro. In addition, recombinant Atsttrin protein effectively attenuated inflammation in several animal models, including collagen antibody- and collagen-induced arthritis models, TNF transgenic mice and dermatitis model (Tang W. et al. (2011), Science, 332: 478-484), (Zhao YP. Et al.,(2013), FEBS Letters, 587: 1805-1810.), indicating that Atsttrin may represent a novel biologics for treating various kinds of TNF/TNFR associated inflammatory diseases and conditions (Tang W. et al. (2011), Science, 332: 478-484; Liu C J et al., (2012), Pharmacology & Therapeutics, 133: 124-132; Liu C J. (2011), FEBS Letters, 585: 3675-3680; Sfikakis P P. et al., (2011), Clin Immunol, 141: 231-235.). Peptides targeting TNF Family receptors, including Atsttrin, are described in the U.S. Pat. No. 8,362,218, the entire contents of which are incorporated herein by reference.

### BRIEF DESCRIPTION OF THE DRAWINGS

FIG. 1. A model illustrating the signaling pathways by which PGRN exerts its actions in normal and diabetic fracture healing. Under normal conditions, PGRN-mediated fracture healing is primarily through the PGRN/TNFR2/14-3-3ε bone regeneration pathway, whereas TNFα/TNFR1 signaling was elevated in diabetic fracture healing (Kayal et al., J Bone Miner Res, 2007. 22(4): p. 560-8.; Kayal et al., J Bone Miner Res, 2010. 25(7): p. 1604-15.; Kayal et al., Bone, 2009. 44(2): p. 357-63). Accordingly, our model contemplates that PGRN-mediated diabetic fracture healing is through 1) activation of PGRN/TNFR2 regeneration pathway and 2) inhibition of the TNFα/TNFR1 bone resorption pathway.

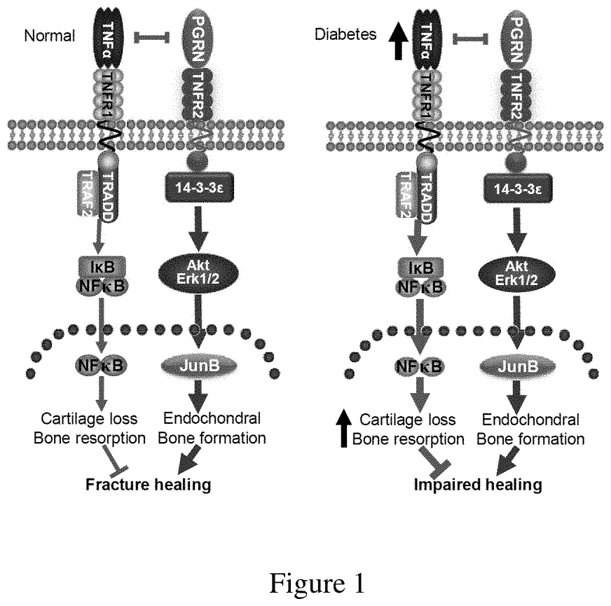

FIG. 2A-2B. (A) Scheme used to identify potential molecules binding to TNFR2 intracellular domains in response to PGRN stimulation. (B) Summary of the hits that were specifically recruited to TNFR2 complexes after treating chondrocytes with PGRN.

FIG. 3A-3B. 14-3-3e binds to TNFR2 in chondrocytes and its expression is differentially regulated by TNFα and PGRN. (A) Co-IP assay. (B) OA chondrocytes were treated with either TNFα or increasing doses of PGRN for 24h and cell lysates were detected with 14-3-3ε or GAPDH antibody.

FIG. 4A-4B. (A) Western blotting confirmed the loss of 14-3-3ε in KO C28I2 cells. (B) PGRN activation of Akt and Erk1/2 was lost in 14-3-3ε KO C28I2. Cells were treated with either vehicle or 200 ng/ml of PGRN followed by immunoblot analysis with indicated antibodies.

FIG. 5A-5D. Loss of PGRN worsened diabetic bone fracture healing. (A) The blood glucose levels in STZ-induced mice and control mice. (B)Representative microCT image of drill-hole model. Red arrows indicate newly-formed callus. (C)Quantification of BV/TV, based on the microCT. (D) HE staining of drill-hole model. Red arrows indicate bone gap. “D” stands for “Diabetes”.

FIG. 6. Recombinant PGRN promoted diabetic bone fracture healing in T1 diabetic mice. Safranin O staining of gravity-induced bone fracture model in diabetic bone fracture healing at various time points.

FIG. 7. rPGRN promotes fracture healing in type 2 diabetic mice. Safranin O staining of gravity-induced bone fracture model in diabetic bone fracture healing at 10 days post operation.

### DETAILED DESCRIPTION OF THE INVENTION

The phrases “impaired bone fracture healing” and “impaired fracture healing” as used herein intend to encompass any anomalies, abnormalities and deficiencies of bone fracture healing, such as inadequate, delayed or absent bone fracture healing, which comprise and denote malunions, delayed unions and non-unions, including, but not limited to, hyperthropic non-unions and atrophic non-unions, pseudarthrosis, hypervascular non-unions (such as elephant foot nonunions, horse hoof nonunions and oligotrophic nonunions) and avascular non-unions (such as torsion wedge nonunions, comminuted nonunions, defect nonunions and atrophic nonunions).

A “malunion” as used herein encompasses a fractured bone that heals in an abnormal position.

A “nonunion” as used herein encompasses a fractured bone that fails to heal after an extended recovery period. In some cases a bone may require up to nine months to completely heal. Nonunion is the cessation of all reparative processes of healing without bony union. Since all of the factors discussed under delayed union usually occur to a more severe degree in nonunion, the differentiation between delayed and nonunion is often based on radiographic criteria and time. In humans, failure to show any progressive change in the radiographic appearance for at least 3 months after the period of time during which normal fracture union would be thought to have occurred, is evidence of nonunion. The changes in radiographic appearance may be slight, and therefore radiographs should be scrutinized monthly to see if, in fact, changes have occurred. The clinical diagnosis of nonunion is usually based on the history and physical findings. Radiographically the diagnosis of nonunion is made by the following findings: a radiolucent line through the fracture site, sealing off of the medullary cavity with sclerosis at the edge of the fractured bone, and bony resorption or regional osteoporosis above and below the fracture site. The bone ends may be somewhat rounded, and a large hypertrophic callus may be present. This “elephant foot” appearance of the callus has been thought of as one of the hallmarks of nonunion. Rarely, an atrophic nonunion is seen without any callus at the fractured bone ends. Sometimes a large gap exists between the ends of the bones while the bones themselves may appear to have little callus formation. This is usually more common when associated with severe soft tissue injury or a loss of vascularity in the area. It may be more common when viewing nonunion after internal fixation and open reduction. When in doubt as to the structural rigidity of the fracture, stress films may be taken to show angular deformities that may occur at the fracture site. Nonunion can occur at any level in any bone.

The term “osteopenia”, as used herein, is defined as a condition in which bone mineral density is lower than normal. It is considered by many doctors to be a precursor to osteoporosis. However, not every person diagnosed with osteopenia will develop osteoporosis. More specifically, osteopenia is defined as a bone mineral density T-score between −1.0 and −2.5. Osteopenia increases the risk of fracture in patients.

The term “poorly oxygenated bone” means a bone with a limited blood supply. Different bones of the skeleton have different amounts of blood supply. Some, such as the bones of the toes, have abundant blood supply; some bones have a moderate blood supply, such as the tibia; while others, such as the head of the femur in the hip joint have a limited blood supply.

The term “smoker” means a subject classified as one or more of the following: current smoker, heavy smoker, and/or cotinine high patient. The term “current smoker” means a subject having ≥100 smoking events in his or her lifetime and smoking within one year prior to commencement of therapy. The term “former smoker” means a subject having ≥100 smoking events in his or her lifetime but has not had any smoking events within the past year. The term “never smoker” means a subject with <100 smoking events in his or her lifetime. The term “ever smoker” means former smokers and current smokers combined. The term “heavy smoker” means a current or former smoker with ≥39 pack-years. The term “light smoker” means a current or former smoker with <39 pack-years.

The term “pack-years” means patient-reported years of smoking times patient reported packs of cigarettes smoked per day. One pack of cigarettes is defined as a commercially available package in the United States with typically 20 cigarettes per peck of any available size or strength. The median number of pack-years for ever smokers described below was 39.

The term “peptide derivatives” encompass and include derivatives to enhance activity, solubility, effective therapeutic concentration, and transport across the blood brain barrier. Further encompassed derivatives include the attachment of moieties or molecules which are known to interact with TNF/TNFR, to target TNF/TNFR or expressing cells, or to have anti-inflammatory activity. The chemical moieties may be N-terminally or C-terminally attached to the peptides of the present invention. Chemical moieties suitable for derivatization may be, for instance, selected from among water soluble polymers. The polymer selected can be water soluble so that the component to which it is attached does not precipitate in an aqueous environment, such as a physiological environment. Preferably, for therapeutic use of the end-product preparation, the polymer will be pharmaceutically acceptable. The polymer may be branched or unbranched. One skilled in the art will be able to select the desired polymer based on such considerations as whether the polymer/component conjugate will be used therapeutically, and if so, the desired dosage, circulation time, resistance to proteolysis, and other considerations. Peptide derivatives and polymers used in modifying the peptides are described in the U.S. Pat. No. 8,362,218, the entire contents of which are incorporated herein by reference.

The term “progranulin derivative” includes a fragment of a progranulin protein as described in WO 2017/024137, as well as a chemically modified progranulin protein as described in the U.S. Pat. No. 8,362,218, the entire contents of which are incorporated herein by reference.

The term “Non Steroidal Anti-Inflammatory Drugs (NSAIDs)”, as used herein, refer to a drug class that groups together drugs that provide analgesic (pain-killing) and antipyretic (fever-reducing) effects, and, in higher doses, anti-inflammatory effects. Examples of NSAIDs include, but are not limited to, Ibuprofen, Aspirin, Ketoprofen, Sulindac, Naproxen, Etodolac, Fenoprofen, Ketorolac, Piroxicam, Indomethacin, Mefenamic Acid, Meloxicam, Nabumetone, Oxaprozin, Ketoprofen, Famotidine, Meclofenamate, Tolmetin, and Salsalate.

In some embodiments, a subject with an impaired bone fracture or fractures is treated with a method comprising administering said subject an effective amount of progranulin or progranulin derivatives. As used herein, “effective amount” of progranulin or progranulin derivatives means an amount sufficient at least to promote impaired bone healing in a subject in need of such treatment.

In some embodiments, an effective amount of progranulin or progranulin derivatives is about 2 μg to 100 μg. In other embodiments, the effective amount of progranulin or progranulin derivatives is about 2 μg, 4 μg, 8 μg, 10 μg, 20 μg, 30 μg, 40 μg, 50 μg, 60 μg, 70 μg, 80 μg, 90 μg or 100 μg of progranulin or progranulin derivatives.

In an embodiment, progranulin or progranulin derivatives can be included in a pharmaceutical composition. As used herein, a pharmaceutical composition is a formulation which contains at least one active ingredient as well as, for example, one or more excipients, buffers, carriers, stabilizers, preservatives and/or bulking agents, and is suitable for administration to a patient to achieve a desired effect or result. The pharmaceutical compositions disclosed herein can have diagnostic, preventative (i.e., phrophylactic), cosmetic and/or research utility in various species, such as for example in human patients or subjects.

This disclosure also provides a composition comprising a combination as described above and a pharmaceutically acceptable carrier. For the purposes of this disclosure, “pharmaceutically acceptable carriers” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, and the like. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

The pharmaceutical preparations of the present disclosure can be made up in any conventional form including, inter alia,: (a) a solid form for oral administration such as tablets, capsules (e.g. hard or soft gelatin capsules), pills, cachets, powders, granules, and the like; (b) preparations for topical administrations such as solutions, suspensions, ointments, creams, gels, micronized powders, sprays, aerosols and the like. The pharmaceutical preparations may be sterilized and/or may contain adjuvants such as preservatives, stabilizers, wetting agents, emulsifiers, salts for varying the osmotic pressure and/or buffers.

The pharmaceutical compositions of the present disclosure can be used in liquid, solid, tablet, capsule, pill, ointment, cream, nebulized or other forms as explained below. In some embodiments, the composition of the present disclosure can be administered by different routes of administration such as oral, oronasal, parenteral or topical.

“Oral” or “peroral” administration refers to the introduction of a substance, such as a vaccine, into a subject's body through or by way of the mouth and involves swallowing or transport through the oral mucosa (e.g., sublingual or buccal absorption) or both.

“Oronasal” administration refers to the introduction of a substance, such as a vaccine, into a subject's body through or by way of the nose and the mouth, as would occur, for example, by placing one or more droplets in the nose. Oronasal administration involves transport processes associated with oral and intranasal administration.

“Parenteral administration” refers to the introduction of a substance, such as a vaccine, into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration. Parenteral administration also includes direct injection into the fracture site. Parenteral administration also includes implantation on a carrier either by injection or open surgical implantation into or on the fracture site. For the purposes of this disclosure, parenteral administration excludes administration routes that primarily involve transport of the substance through mucosal tissue in the mouth, nose, trachea, and lungs.

Formulations suitable for parenteral administration comprise a composition comprising progranulin or progranulin derivatives in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e.g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a composition comprising progranulin or progranulin derivatives, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered formulation is accomplished by dissolving or suspending the composition comprising progranulin or progranulin derivatives in an oil vehicle.

Injectable depot forms are made by forming microencapsule matrices of a composition comprising progranulin or progranulin derivatives or in biodegradable polymers such as polylactide-polyglycolide. Depending on the ratio of the composition comprising progranulin or progranulin derivatives to polymer, and the nature of the particular polymer employed, the rate of drug release can be controlled. Examples of other biodegradable polymers include poly (orthoesters) and poly (anhydrides). Depot injectable formulations are also prepared by entrapping a composition comprising progranulin or progranulin derivatives in liposomes or microemulsions which are compatible with body tissue.

“Topical administration” means the direct contact of a substance with tissue, such as skin or membrane, particularly the oral or buccal mucosa.

For topical administration to the skin or mucous membrane the aforementioned composition is preferably prepared as ointments, tinctures, creams, gels, solution, lotions, sprays; aerosols and dry powder for inhalation, suspensions, shampoos, hair soaps, perfumes and the like. In fact, any conventional composition can be utilized in this invention. Among the preferred methods of applying the composition containing the agents of this invention is in the form of an ointment, gel, cream, lotion, spray; aerosol or dry powder for inhalation. The pharmaceutical preparation for topical administration to the skin can be prepared by mixing the aforementioned active ingredient with non-toxic, therapeutically inert, solid or liquid carriers customarily used in such preparation. These preparations generally contain 0.01 to 5.0 percent by weight, or 0.1 to 1.0 percent by weight, of the active ingredient, based on the total weight of the composition.

In preparing the topical preparations described above, additives such as preservatives, thickeners, perfumes and the like conventional in the art of pharmaceutical compounding of topical preparation can be used. In addition, conventional antioxidants or mixtures of conventional antioxidants can be incorporated into the topical preparations containing the aforementioned active agent. Among the conventional antioxidants which can be utilized in these preparations are included N-methyl-a-tocopherolamine, tocopherols, butylated hydroxyanisole, butylated hydroxytoluene, ethoxyquin and the like.

Cream-based pharmaceutical formulations containing the active agent, used in accordance with this invention, are composed of aqueous emulsions containing a fatty acid alcohol, semi-solid petroleum hydrocarbon, ethylene glycol and an emulsifying agent.

Ointment formulations containing the active agent in accordance with this invention comprise admixtures of a semi-solid petroleum hydrocarbon with a solvent dispersion of the active material. Cream compositions containing the active ingredient for use in this invention preferably comprise emulsions formed from a water phase of a humectant, a viscosity stabilizer and water, an oil phase of a fatty acid alcohol, a semi-solid petroleum hydrocarbon and an emulsifying agent and a phase containing the active agent dispersed in an aqueous stabilizer-buffer solution. Stabilizers may be added to the topical preparation. Any conventional stabilizer can be utilized in accordance with this invention. In the oil phase, fatty acid alcohol components function as a stabilizer. These fatty acid alcohol components function as a stabilizer. These fatty acid alcohol components are derived from the reduction of a long-chain saturated fatty acid containing at least-14 carbon atoms. The ointments, pastes, creams and gels may contain, in addition to progranulin or progranulin derivatives, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a composition

WO 2018/136680 PCT/US2018/014324 comprising progranulin or progranulin derivatives, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Also, conventional perfumes and lotions generally utilized in topical preparation for the hair can be utilized in accordance with this invention. Furthermore, if desired, conventional emulsifying agents can be utilized in the topical preparations of this invention.

An oral dosage form comprises cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, or capsules of hard or soft gelatin, methylcellulose or of another suitable material easily dissolved in the digestive tract, each containing a predetermined amount of a composition comprising progranulin or progranulin derivatives as an active ingredient. Each tablet, pill, sachet or capsule can preferably contain from about progranulin or progranulin derivatives as active ingredient. The oral dosages contemplated in accordance with the present invention will vary in accordance with the needs of the individual patient as determined by the prescribing physician.

A compound may also be administered as a bolus, electuary, or paste.

In solid dosage forms for oral administration (e.g., capsules, tablets, pills, dragees, powders, granules and the like), a composition comprising progranulin or progranulin derivatives is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate, (5) solution retarding agents, such as paraffin, (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of a composition comprising progranulin or progranulin derivatives therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the composition comprising progranulin or progranulin derivatives only, or preferentially, in a certain portion of the gastrointestinal tract, optionally, in a delayed manner Examples of embedding compositions which can be used include polymeric substances and waxes. The composition comprising progranulin or progranulin derivatives can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to a composition comprising progranulin or progranulin derivatives, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to a composition comprising progranulin or progranulin derivatives, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compositions comprising progranulin or progranulin derivatives with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Formulations which are suitable for vaginal administration also include pessaries, tampons, creams, gels, pastes, foams or spray formulations containing such carriers as are known in the art to be appropriate.

Compositions comprising progranulin or progranulin derivatives can be alternatively administered by aerosol. For example, this can be accomplished by preparing an aqueous aerosol, liposomal preparation or solid particles containing a composition comprising progranulin or progranulin derivatives preparation. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound Accordingly, a feature of the present invention is the provision of an osteogenic composition in the form of a sponge that includes a substantial amount of a relatively slowly-resorbed mineral component that remains at the implant site after the carrier has been rapidly resorbed, in order to provide a scaffold for new bone formation that is not prematurely resorbed due to the osteoclastic potentiation by the bone morphogenic protein in the composition. The present invention also provides methods for using such osteogenic compositions in treatment of bone trauma, disease and defects, for artificial arthrodeses and for other treatment where new bone formation is desired, especially in primates, including humans.

The sponge matrix material is preferably collagenous. A wide variety of collagen materials are suitable for the sponge matrix. Naturally occurring collagens may be subclassified into several different types depending on their amino acid sequence, carbohydrate content and presence or absence of disulfide cross-links. Types I and III collagen are two of the most common subtypes of collagen. Type I collagen is present in skin, tendon and bone whereas Type III collagen is found primarily in skin. The collagen in the composition may be obtained from skin, bone, tendon, or cartilage and purified by methods known in the art. Alternatively, the collagen may be purchased commercially. The collagen in the composition is preferably Type I bovine collagen.

The collagen carrier can further be atelopeptide collagen and/or telopeptide collagen. Moreover, both non-fibrillar and fibrillar collagen may be used. Non-fibrillar collagen is collagen that has been solubilized and has not been reconstituted into its native fibrillar form.

The sponge carrier may also be formed of other natural or synthetic polymeric materials, in addition to or as an alternative to collagen. For example, the sponge carrier may be formed of gelatin (e.g. foamed gelatin), in addition collagen or as an alternative to collagen. Other natural and synthetic polymers are also known for the formation of biocompatible sponge materials, and can be used herein. typically include nonionic surfactants, innocuous proteins like serum albumin, sorbitan esters, oleic acid, lecithin, amino acids such as glycine, buffers, salts, sugars or sugar alcohols. Aerosols generally are prepared from isotonic solutions.

In another embodiment, compositions comprising progranulin or progranulin derivatives are delivered locally by a sponge matrix material. Accordingly, a feature of the present disclosure is the provision of an osteogenic composition comprising progranulin or progranulin derivatives in the form of a sponge that includes a substantial amount of a relatively slowly-resorbed mineral component.

The sponge matrix material is preferably collagenous. A wide variety of collagen materials are suitable for the sponge matrix. Naturally occurring collagens may be subclassified into several different types depending on their amino acid sequence, carbohydrate content and presence or absence of disulfide cross-links. Types I and III collagen are two of the most common subtypes of collagen. Type I collagen is present in skin, tendon and bone whereas Type III collagen is found primarily in skin. The collagen in the composition may be obtained from skin, bone, tendon, or cartilage and purified by methods known in the art. Alternatively, the collagen may be purchased commercially. In a specific embodiment, the collagen in the composition is Type I bovine collagen.

The collagen carrier can further be atelopeptide collagen and/or telopeptide collagen. Moreover, both non-fibrillar and fibrillar collagen may be used. Non-fibrillar collagen is collagen that has been solubilized and has not been reconstituted into its native fibrillar form.

The sponge carrier may also be formed of other natural or synthetic polymeric materials, in addition to or as an alternative to collagen. For example, the sponge carrier may be formed of gelatin (e.g. foamed gelatin), in addition collagen or as an alternative to collagen. Other natural and synthetic polymers are also known for the formation of biocompatible sponge materials, and can be used herein.

In another embodiment, said subject is suffering from a disease that causes impaired bone fracture healing, such as diabetes, liver disease, Cushing's disease or other metabolic diseases,

In yet another embodiment, said subject is a smoker.

In another embodiment, said subject is using Non Steroidal Anti-Inflammatory Drugs (NSDAIDS). In a specific embodiment, the NSAID is selected from the group consisting of Ibuprofen, Aspirin, Ketoprofen, Sulindac, Naproxen, Etodolac, Fenoprofen, Ketorolac, Piroxicam, Indomethacin, Mefenamic Acid, Meloxicam, Nabumetone, Oxaprozin, Ketoprofen, Famotidine, Meclofenamate, Tolmetin, and Salsalate.

In a specific embodiment said progranulin derivative is Attstrin (SEQ ID NO: 3).

In another aspect of the present disclosure, progranulin or progranulin derivatives are used in a method of treating impaired bone fracture. In a specific embodiment progranulin or progranulin derivatives are administered to a subject suffering from impaired bone fractures at a dose about 2 μg to 100 μg based on the route of administration. In other embodiments, the progranulin or progranulin derivatives is administered at a dose about 2 μg, 4 μg, 8 μg, 10 μg, 20 μg, 30 μg, 40 μg, 50 μg, 60 μg, 70 μg, 80 μg, 90 μg or 100 μg of progranulin or progranulin derivatives.

In yet another embodiment, progranulin or progranulin derivatives are administered systemically to a patient in need thereof every day, every two days or every three days. In a specific embodiment progranulin or progranulin derivatives are administered once a week. Administration of progranulin or progranulin derivatives is continued until desired bone repair outcome is observed. Bone repair status is monitored in such subjects by X-Ray or other suitable imaging techniques.

In a specific embodiment, progranulin or progranulin derivatives are administered to the bone fracture site locally via a collagen sponge comprising 2 μg, 4 μg, 8 μg, 10 μg, 20 μg, 30 μg, 40 μg, 60 μg, 70 μg, 80 μg, 90 μg or 100 μg progranulin or progranulin derivatives as an active compound. Collagen sponges can be surgically placed at the bone fracture site once, or can be replaced with new progranulin or progranulin derivative-containing sponges as necessary.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Surgical Procedure

All animals were provided with water and food ad libitum throughout the duration of the study. In all in vivo experiments mice were anesthetized with a mixture of xylazine and ketamine (0.15% xylazine and 0.85% ketamine), which was injected intraperitoneally at 1 μl/g body weight, and operations were performed (table 1).

Segmental femoral bone defect model—The method by which we created the segmental femoral bone defect model in WT and diabetic mice (db/db) was based on a previously described protocol with modifications (Lin et al. J Orthop Trauma, (2012); 26:719-723). A 1cm skin incision was made overlying the lateral right femur and lateral femur was exposed. Through the same incision, the knee joint was exposed using a medial parapatellar approach, and the patella was dislocated laterally. A 1.5-inch 25-gauge needle was then inserted between the femoral condyles. A tranverse osteotomy was made through the mid-femoral shaft using sharp surgical scissors. Fractures with significant comminution were excluded. Using a 0.75 mm drill bit and motorized drill, two holes were then placed in an anteroposterior direction at 2.2 mm from the fractured ends. The femur was then stabilized by advancing the needle that was then cut. A fracture gap of 0.5 mm was established by inserting a custom 5 mm metallic clip into the previously drilled holes. The wound was then irrigated with saline. The muscle and skin were then closed. A dissecting microscope was used throughout the operation.

The femoral drill-hole model—The drill-hole model in the femur was created as described previously (He et al., Bone; (2011); 48:1388-1400.). Briefly, unicortical holes of 0.8 mm in diameter were generated in WT and diabetic mice (db/db) through anterior cortices using a 21-gauge needle. Holes were rinsed by injection of saline to remove bone fragments from the cavity. Thereafter, the mice from both genotypes were sacrificed on days 0, 5, 15 and 21 (n=6 for each group) post injury.

Nonunion segmental radial defect model—The method by which we created a nonunion segmental radial defect model was based on a previously described protocol (Kimelman-Bleich et al., (2009), Biomaterials. 30:4639-4648.).

Briefly, male WT and db/db (diabetic) mice were anesthetized in the manner described above. The skin was cut and a 1.5mm long defect was created in the right radius bone. A 1.5 mm long collagen sponge (a kind gift of Dr. Gino Bradica, Kensey Nash Corp. Exton, Pa.) was then implanted into the defect and the skin was sutured. For WT mice, the animals were randomly allocated to the following groups: (i) PBS (n=6); (ii) recombinant PGRN (4 μg, n=6); (iii) recombinant PGRN (8 μg, n=6). For diabetic mice (db/db), the animals were randomly allocated to the following groups: (i) PBS (n=6); (ii) recombinant PGRN (8 μg, n=6). All of the reagents were diluted into 10 μl PBS, and dripped slowly and gently into the collagen sponge to make them distribute well, and all of the

### Example 1

### Progranulin Binds TNFα Receptor TFNR2 with High Affinity and Promotes Bone Healing in a Diabetic Bone Healing Model

The present inventors discovered that PGRN has an approximately 600-fold higher binding affinity for TFNR2 than TNFα (K_{D} of PGRN vs. TNFα: 1.52×10⁻⁹M vs. 9.10×10⁻⁷ M). The present inventors also have demonstrated that TNFR2 is critical for PGRN-mediated fracture healing. Importantly, a combination of biochemical co-purification with highly sensitive mass spectrometry has revealed that 14-3-3ε, a critical signaling mediator of TLR signaling, is a novel component of the PGRN/TNFR2 complex. In brief, PGRN exerts its role in diabetic bone healing process via at least two pathways: 1) by antagonizing TNF/TNFR1 catabolic signaling, and 2) by activating the PGRN/TNFR2/14-3-3ε protective pathway.

### Example 2

### Progranulin Promotes Bone Healing in a Diabetic Impaired Bone Healing Model

To determine whether treatment with recombinant PGRN can promote bone regeneration in an impaired bone healing model, a radial bone defect model was established in WT and diabetic mice as described previously (Kimelman-Bleich et al., (2009), Biomaterials. 30:4639-4648.). Collagen sponges with PBS or different doses of recombinant PGRN were implanted into the bone defect location. Fourteen days later, mice were sacrificed and radius samples were collected and analyzed. There was hardly any new bone formation in PBS control group, while PGRN groups displayed markedly enhanced bone regeneration.

### Example 3

### 14-3-3ε was Identified as a Component of TNFR2 Complex in Response to PGRN Treatment

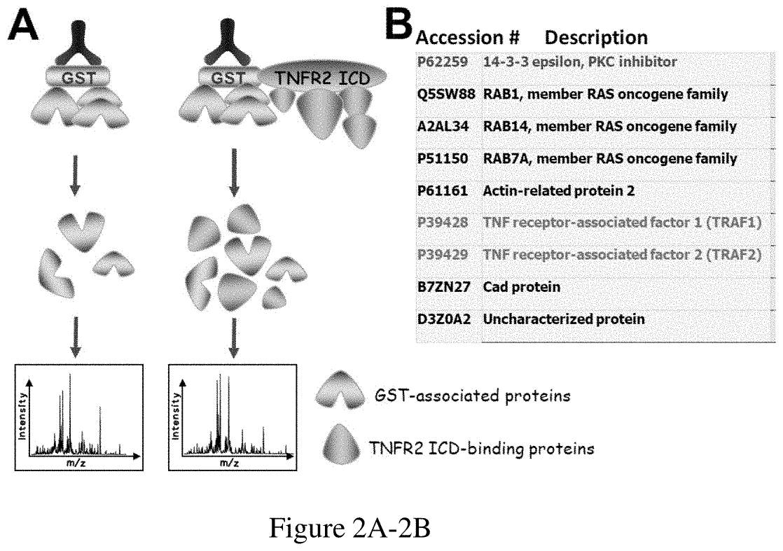

The finding that TNFR2 is the major receptor for mediating PGRN activity in fracture healing prompted the present inventors to explore the intracellular co-factor(s) of TNFR2 that relay the PGRN signaling. To isolate such co-factor(s), the intracellular domain (ICD) of TNFR2 was cloned into the PGEX-3X vector to express a fusion of GST to TNFR2ICD. As illustrated in FIG. 2A, GST (serving as a control) or GST-TNFR2ICD was affinity-purified on glutathione-agarose beads and used as a bait to trap proteins from PGRN-treated human C28I2 chondrocytes. These samples were then analyzed by mass spectrometry, with MS/MS spectra searched against the Uniprot database, using Sequest within Proteome Discoverer. After subtracting the hits that were also trapped by the GST column, we found nine proteins that specifically bind to TNFR2 (FIG. 2B). Identification of TRAF1 and TRAF2 (two known TNFR-binding proteins) among the nine hits validated the technique. The protein that ranked first was 14-3-3ε, a signaling molecule known to be a critical mediator of the Toll-like Receptors (TLRs) pathway.

### Example 4

### Treatment with PGRN, but not TNFα, Recruits 14-3-3ε to the TNFR2 Complex in Chondrocytes

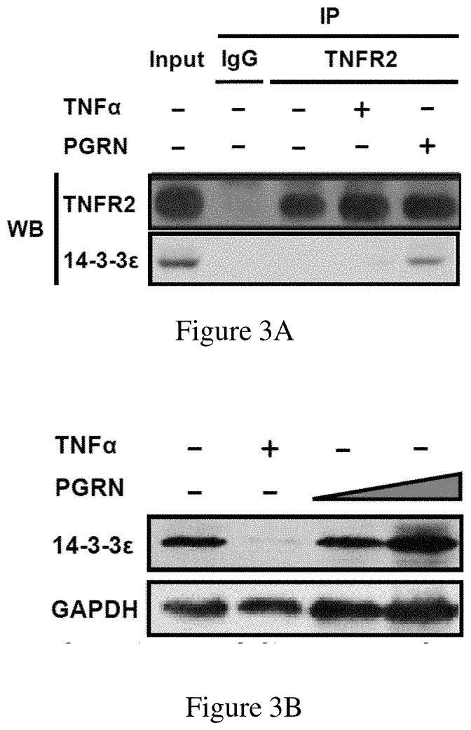

The inventors next used a co-immunoprecipitation (CoIP) assay to confirm the association of 14-3-3ε and TNFR and determined whether this association was affected by PGRN or TNFα treatment. Briefly, chondrocytes were treated with either 10 ng/ml of TNFα or 100 ng/ml of PGRN for 30 min, and cell lysates were immunoprecipitated with TNFR2 antibody followed by immunoblot analysis with the antibodies indicated. As shown in FIG. 3A, 14-3-3ε was specifically detectable in the PGRN-treated cell extracts, clearly indicating that 14-3-3ε was recruited to the TNFR2 complex following PGRN treatment. Interestingly, PGRN treatment increased, whereas TNFα decreased, the level of 14-3-3ε in chondrocytes (FIG. 3B), suggesting that 14-3-3ε may act as a signaling switch of TNFR2 in response to PGRN and TNFα stimulation.

### Example 5

### Knockout of 14-3-3ε Abolishes PGRN-Mediated Activation of AKT and Erk1/2 Signaling in Chondrocytes

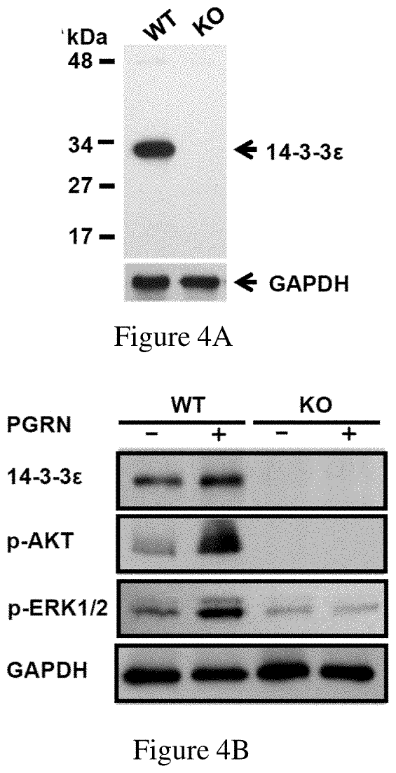

The finding that PGRN treatment recruited 14-3-3ε to the TNFR2 complex led the inventors to determine whether PGRN-activated signaling depends on the presence of 14-3-3ε. For this purpose, the inventors first deleted the 14-3-3ε gene in the C28I2 chondrocyte line by using CRISPR/Cas9 technology, a powerful genome-editing approach. The knockout of 14-3-3ε in C28I2 chondrocytes was confirmed using Immunoblotting with anti-14-3-3E antibody (FIG. 4A). PGRN is known to activate AKT and Erk1/2 signaling in chondrocytes (Feng et al., FASEB J, 2010. 24(6): p. 1879-92.), and the activation of these pathways by PGRN was lost in 14-3-3ε KO chondrocytes (FIG. 4B). These in vitro results indicated that 14-3-3ε is a critical mediator of PGRN/TNFR2 signaling.

### Example 6

### Loss of PGRN Further Delays Diabetic Fracture Healing

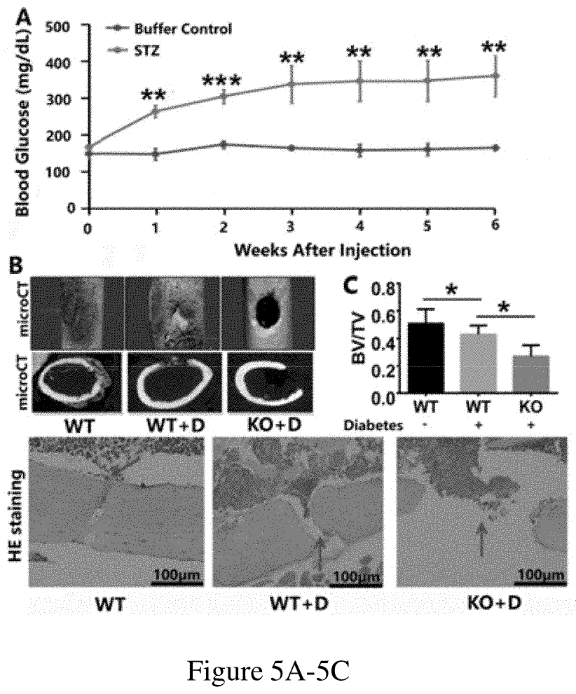

The inventors next performed a study to determine whether endogenously expressed PGRN is important for diabetic fracture healing. To do so, we generated PGRN deficient diabetic mice and established a drill-hole bone healing model. Briefly, the mice were injected daily with phosphate buffered saline (PBS) or STZ (50 mg/kg) for 5 days. Mice were considered diabetic when blood glucose levels exceeded 250 mg/dl. As revealed in FIG. 5A, the blood glucose levels were dramatically higher in STZ-induced mice than in control mice at various time points. Importantly, bone healing was almost entirely lost in the PGRN-deficient diabetic fracture model in a drill-hole bone healing model, assayed by microCT and histological staining (FIGS. 5B-D), indicating that endogenous PGRN is critical for diabetic fracture healing.

### Example 7

### Recombinant PGRN Promotes Diabetic Done Fractures in Type 1 Diabetic Mice

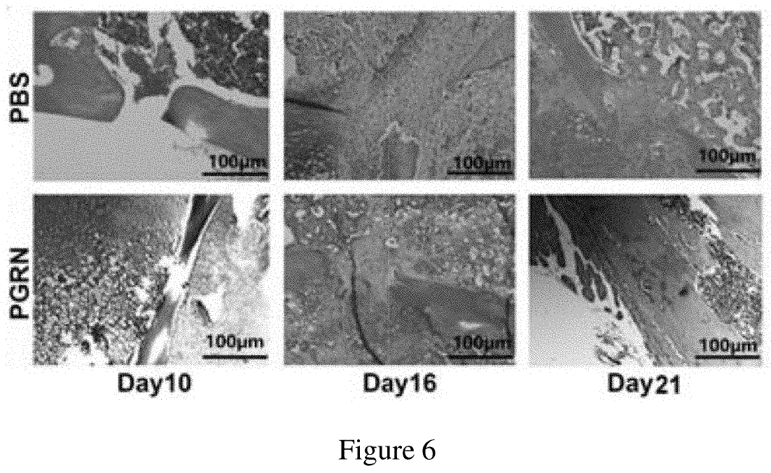

Revelation of the important role of endogenous PGRN in fracture healing led the present inventors to determine whether recombinant PGRN could enhance diabetic fracture healing. For this purpose, the inventors performed studies in which the gravity-induced bone fracture model was established in type 1 diabetic mice, and recombinant PGRN, at a dose of 10 μg/g body weight, was injected intraperitoneally (IP) twice a week. As shown in FIG. 6, recombinant PGRN effectively accelerated chondrogenesis and callus formation, leading to enhanced diabetic bone regeneration in gravity-induced bone fracture.

### Example 8

### Recombinant PGRN Promotes Diabetic Bone Fractures in Type 2 Diabetic Mice

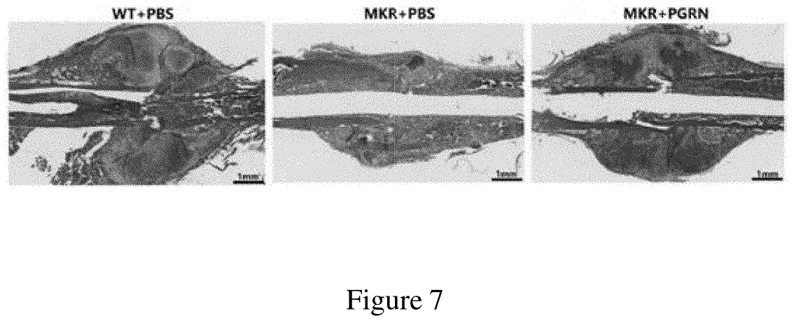

The inventors also tested the role of PGRN in the type 2 diabetic mice, and have performed a study on type 2 diabetic mice. The inventors took advantage of genetically modified MKR mice, which are used to study type 2 diabetes (provided by Dr. Xin Li from NYU School of Dentistry). The mice were sacrificed at 10 days post operation and the callus formation was analyzed by Safranin O staining. As revealed in FIG. 7, the diabetic condition caused a much smaller callus, but this dramatic reduction in callus formation could be effectively reversed by intraperitoneal (IP) injection of rPGRN (at a dosage of 10 μg/g body weight).

What is claimed is:

1. A method of treating impaired bone fracture healing comprising administering to a subject in need of such treatment an effective amount of progranulin or a progranulin derivative.

2. The method in claim 1, wherein said subject is diabetic.

3. The method in claim 1, wherein said subject has liver disease.

4. The method in claim 1, wherein said subject is a smoker.

5. The method in claim 1, wherein said subject is on a NSAID treatment.

6. The method in claim 1, wherein said subject has Cushing's disease.

7. The method of claim 1, wherein said progranulin derivative is a progranulin fragment.

8. The method of claim 1, wherein said progranulin derivative is a chemically-modified progranulin.

9. The method in claim 1, wherein said progranulin derivative is Attstrin (SEQ ID NO:
3).

10. The method in claim 1, wherein said impaired bone fracture healing is selected from the group consisting of a delayed union, a non-union bone fracture, and a fracture of a poorly oxygenated bone.

11. The method in claim 1, wherein said progranulin or progranulin derivative is administered to said subject once a week until desired bone repair is observed.

12. The method in claim 1, wherein said progranulin or progranulin derivatives are administered to said subject with a collagen sponge carrier.


SEQUENCE LISTING (3 sequences)

SEQ ID NO: 1 - DNA, 1779 bp, Homo sapiens
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctg

SEQ ID NO: 2 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - protein, 154 aa, Artificial Sequence
  Synthesized Polypeptide
PQASCCEDRVHCCPHGAFCDLVHTRCITPTGTHPLAKKLPAQRTNRAVALSSSSKENATTDLLTKLPAHTVGDVKCDMEVSCPDGYTCCRLQSGAWCEQGPHQVPWMEKAPAHLSLPDPQALKRDVPCDNVSSCPSSDTCCQLTSGEWGCCPIP